(19) United States
(12) Patent Application Publication
(10) Pub. No.: US 2025/0006365 A1
(43) Pub. Date: Jan. 2, 2025

(54) Disease Analysis Method, Training Method and Apparatus of Disease Analysis Model

(71) Applicant: BOE Technology Group Co., Ltd., Beijing (CN)

(72) Inventors: Yang SONG, Beijing (CN); Ding DING, Beijing (CN)

(21) Appl. No.: 18/275,018

(22) PCT Filed: Jul. 29, 2022
(86) PCT No.: PCT/CN2022/109023

§ 371 Date: Jul. 31, 2023

(51) Int. Cl. G16H 50/20; G16H 50/50; G16H 50/70

(52) U.S. Cl. CPC G16H 50/20; G16H 50/50; G16H 50/70

(57) ABSTRACT

Disclosed are a disease analysis method, a training method and apparatus of a disease analysis model, the disease analysis method includes: acquiring first omics data and second omics data of a patient, wherein first omics includes a plurality of first loci and second omics includes a plurality of second loci; inputting the first omics data and the second omics data into a fusion algorithm model to obtain a predicted result of the patient, wherein the fusion algorithm model is formed by fusion and construction according to a first omics model and a second omics model, the first omics model is constructed according to sample data of the first omics, and the second omics model is constructed according to sample data of the second omics.

### CROSS-REFERENCE TO RELATED APPLICATION

The present application is a U.S. National Phase Entry of International Application No. PCT/CN2022/109023 having an international filing date of Jul. 29, 2022. The entire contents of the above-identified application are hereby incorporated by reference.

### TECHNICAL FIELD

Embodiments of the present disclosure relate to, but are not limited to, the technical field of biological information, in particular to a disease analysis method, a training method and apparatus of a disease analysis model.

### BACKGROUND

Some tumors have relatively strong invasiveness and prognosis of patients is poor. How to improve accuracy of the prognosis of patients, establish an effective method evaluation system, and provide personalized treatment guidance to patients is a very concerned issue in today's national policy layout and scientific research field. A development mechanism of a tumor is complex, which is influenced by multiple factors such as gene level and appearance level. DeoxyriboNucleic Acid (DNA) methylation and gene mutation are closely related to occurrence and development of the tumor.

At present, prognostic methods of tumors have following problems: 1) factors considered in an evaluation system of monoomics are limited and comprehensive evaluation cannot be performed; an evaluation system of multiomics is simple and integrated, which does not give full play to conditional advantages of multiple factors, all of which will have a certain impact on accuracy of evaluation; 2) patients with similar clinical manifestations are not effectively associated, and patients with mutual connection may have consistency in disease diagnosis and treatment and prognosis.

### SUMMARY

The following is a summary of subject matters described herein in detail. The summary is not intended to limit the protection scope of claims.

An embodiment of the present disclosure provides a disease analysis method, including: acquiring first omics data and second omics data of a patient, wherein first omics includes a plurality of first loci and second omics includes a plurality of second loci; and inputting the first omics data and the second omics data into a fusion algorithm model to obtain a predicted result of the patient, wherein the fusion algorithm model is formed by fusion and construction according to a first omics model and a second omics model, the first omics model is constructed according to sample data of the first omics, and the second omics model is constructed according to sample data of the second omics.

An embodiment of the present disclosure further provides a disease analysis apparatus, including a memory and a processor connected to the memory, wherein the memory is configured to store instructions, and the processor is configured to execute acts of the disease analysis method according to any embodiment of the present disclosure based on the instructions stored in the memory.

An embodiment of the present disclosure also provides a computer-readable storage medium having stored thereon a computer program, wherein, when the program is executed by a processor, the disease analysis method according to any embodiment of the present disclosure is implemented.

An embodiment of the present disclosure also provides a training method of a disease analysis model, including: performing data preprocessing on sample data of first omics and sample data of second omics respectively; performing feature filtering on the sample data of the first omics and the sample data of the second omics respectively; calculating a first similarity matrix between the sample data of the first omics, and constructing a first undirected link network according to the calculated first similarity matrix; calculating a second similarity matrix between the sample data of the second omics, and constructing a second undirected link network according to the calculated second similarity matrix; constructing a first omics model according to the constructed first undirected link network and eigenvalues of the first omics; constructing a second omics model according to the constructed second undirected link network and eigenvalues of the second omics; multiplying probability values with a same predicted category in the first omics model and the second omics model to obtain a product probability matrix; and constructing a fusion algorithm model according to a two-dimensional undirected link network and the product probability matrix, wherein the two-dimensional undirected link network is the first undirected link network or the second undirected link network.

An embodiment of the present disclosure also provides a training apparatus of a disease analysis model, including a memory and a processor connected to the memory, wherein the memory is configured to store instructions, and the processor is configured to execute acts of the training method of the disease analysis model according to any embodiment of the present disclosure based on the instructions stored in the memory.

An embodiment of the present disclosure also provides a computer-readable storage medium having stored thereon a computer program, wherein, when the program is executed by a processor, the training method of the disease analysis model according to any embodiment of the present disclosure is implemented.

Other aspects may be comprehended upon reading and understanding drawings and detailed description.

### BRIEF DESCRIPTION OF DRAWINGS

Accompanying drawings are used for providing further understanding of technical solutions of the present disclosure, constitute a part of the specification, and together with the embodiments of the present disclosure, are used for explaining the technical solutions of the present disclosure, but do not constitute limitations on the technical solutions of the present disclosure. Shapes and sizes of various components in the drawings do not reflect actual scales, but are only intended to schematically illustrate contents of the present disclosure.

FIG. 1 is a schematic flowchart of a disease analysis method according to an exemplary embodiment of the present disclosure.

FIG. 2A is a schematic diagram of a methylated data set after data preprocessing according to an exemplary embodiment of the present disclosure.

FIG. 2B is a schematic diagram of a gene mutation data set after data preprocessing according to an exemplary embodiment of the present disclosure.

FIG. 3A is a schematic diagram of a methylation data set after low variance feature filtering according to an exemplary embodiment of the present disclosure.

FIG. 3B is a schematic diagram of a gene mutation data set after low variance feature filtering according to an exemplary embodiment of the present disclosure.

FIG. 4A is a schematic diagram of a methylated data set after low variance feature filtering and recursive dimension reduction feature filtering according to an exemplary embodiment of the present disclosure.

FIG. 4B is a schematic diagram of a gene mutation data set after low variance feature filtering and recursive dimension reduction feature filtering according to an exemplary embodiment of the present disclosure.

FIG. 5A is a schematic diagram of a first undirected link network relationship data set obtained according to the methylated data set of FIG. 3A.

FIG. 5B is a schematic diagram of a second undirected link network relationship data set obtained according to the gene mutation data set of FIG. 3B.

FIG. 5C is a network diagram of a first undirected link network or a second undirected link network that has been constructed.

FIG. 6A is a Receiver Operating Characteristic (ROC) curve graph of a methylation algorithm model constructed according to the first undirected link network relationship data set of FIG. 5A.

FIG. 6B is a ROC curve graph of a gene mutation algorithm model constructed according to the second undirected link network relationship data set of FIG. 5B.

FIG. 7A is a ROC curve graph of a methylation algorithm model constructed according to a first undirected link network relationship methylation data set of FIG. 4A after low variance feature filtering and recursive dimension reduction feature filtering.

FIG. 7B is a ROC curve graph of a gene mutation algorithm model constructed according to a second undirected link network relationship gene mutation data set of FIG. 4B after low variance feature filtering and recursive dimension reduction feature filtering.

FIG. 8 is a ROC curve graph of a fusion algorithm model created according to the methylation algorithm model of FIG. 7A and the gene mutation algorithm model of FIG. 7B.

FIG. 9A is a schematic diagram of a clinical molecular marker system of top 10 of methylation obtained according to a disease analysis method of an embodiment of the present disclosure.

FIG. 9B is a schematic diagram of a clinical molecular marker system of top 10 of gene mutation obtained according to a disease analysis method of an embodiment of the present disclosure.

FIGS. 10A and 10B are schematic flowcharts of two other disease analysis methods according to exemplary embodiments of the present disclosure.

FIG. 11 is a schematic diagram of a structure of a disease analysis apparatus according to an exemplary embodiment of the present disclosure.

FIG. 12 is a schematic flowchart of a training method of a disease analysis model according to an exemplary embodiment of the present disclosure.

### DETAILED DESCRIPTION

To make objectives, technical solutions, and advantages of the present disclosure clearer, the embodiments of the present disclosure will be described in detail with reference to the accompanying drawings. It needs to be noted that the embodiments in the present disclosure and features in the embodiments may be randomly combined with each other if there is no conflict.

Unless otherwise defined, technical terms or scientific terms publicly used in the embodiments of the present disclosure should have usual meanings understood by those of ordinary skill in the art to which the present disclosure belongs. “First”, “second”, and similar terms used in the embodiments of the present disclosure do not represent any order, quantity, or importance, but are only used for distinguishing different components. “Include”, “contain”, or a similar term means that an element or article appearing before the term covers an element or article and equivalent thereof listed after the term, and other elements or articles are not excluded.

As shown in FIG. 1, an embodiment of the present disclosure provides a disease analysis method, including following acts.

Act 101: acquiring first omics data and second omics data of a patient, wherein first omics includes a plurality of first loci and second omics includes a plurality of second loci.

Act 102: inputting the first omics data and the second omics data into a fusion algorithm model to obtain a predicted result of the patient, wherein the fusion algorithm model is formed by fusion and construction according to a first omics model and a second omics model, the first omics model is constructed according to sample data of the first omics, and the second omics model is constructed according to sample data of the second omics.

According to the embodiment of the present disclosure, a fusion algorithm model is established according to the first omics model and the second omics model and a disease is predicted according to the fusion algorithm model, disease diagnosis and treatment or prognosis of the patient can be more accurately determined, avoiding problems that comprehensive evaluation cannot be performed in an evaluation system of monoomics and an evaluation system of multiomics cannot effectively play advantages of multi-factor conditions. The disease described in the embodiment of the present disclosure may be a tumor or another disease, which is not limited in the embodiment of the present disclosure.

In the embodiment of the present disclosure, omics mainly includes DNA methylation omics, genomics, proteomics, and transcriptomics, etc.

In some exemplary implementation modes, a first locus may be a DNA methylation locus, and a second locus may be a gene locus; or, the first locus may be a gene locus, and the second locus may be a DNA methylation locus, wherein information that may be included in the gene locus may include a mutation situation and/or an expression situation, however, which is not limited in the embodiment of the present disclosure.

Exemplarily, the first locus may be a DNA methylation locus, and the second locus may be a gene locus, information included in the gene locus is gene mutation situation information. Within a biological system, methylation is catalyzed by enzymes, and the methylation involves heavy metal modification, regulation of gene expression, adjustment of protein function, and ribonucleic acid processing. Change of methylation is usually related to abnormal expression of disease genes. A specific position occupied by a gene on chromosome is called a gene locus. A quantity of genes is large, but a quantity of chromosomes is relatively small, so one chromosome contains many genes, and the genes are arranged in a single line on the chromosome. Gene mutation refers to change of a gene in a cell, which includes point mutation of single base, repetition, insertion, and deletion of multiple bases, etc. Gene mutation will lead to changes in protein expression, thus affecting cellular function adversely.

In some exemplary implementation modes, the first omics model is constructed according to the sample data of the first omics, which may include: performing data preprocessing on the sample data of the first omics; performing feature filtering on the sample data of the first omics; calculating a first similarity matrix between the sample data of the first omics, and constructing a first undirected link network according to the calculated first similarity matrix; and constructing the first omics model according to the constructed first undirected link network and eigenvalues of the first omics.

In the embodiment of the present disclosure, both the first undirected link network and a second undirected link network described later are two-dimensional undirected link networks. A two-dimensional undirected link network is a two-dimensional network composed of connecting lines between nodes. Nodes with similar data angles will be connected using undirected line segments, and data characteristics of surrounding nodes will be considered comprehensively in model construction and prediction of unknown nodes. According to the embodiment of the present disclosure, a local network of patients with internal connection is established through a two-dimensional undirected link network, a similar diagnosis and treatment scheme can be implemented in a targeted manner for patients in the local network, and prognosis of patients can be determined more effectively.

In some exemplary implementation modes, performing data preprocessing on the sample data of the first omics may include: deleting one or more first loci in the sample data of the first omics, wherein there is no data value for at least one patient sample at each deleted first locus.

Exemplarily, assuming that the first omics includes a plurality of methylation loci, when performing data preprocessing on sample data of the methylation loci, a methylation locus is deleted when there is one or more patient samples at the methylation locus where no methylation level value is detected.

In some exemplary implementation modes, performing data preprocessing on the sample data of the first omics may include: deleting one or more first loci in the sample data of the first omics, wherein there is no data value for at least b % of patient samples at each deleted first locus, and b is a real number greater than 0; and performing data filling on a patient sample with no data value in the sample data of the first omics.

Exemplarily, still taking a case where the first omics includes a plurality of methylation loci as an example, if b=20, when performing data preprocessing on sample data of the methylation loci, a methylation locus is deleted when there are more than 20% of patient samples at the methylation locus where no methylation level value is detected. If there is missing data at a reserved methylation locus, a median value or average value of the methylation locus is used for filling to obtain a final methylation locus and form a methylation data set.

In some exemplary implementation modes, performing data preprocessing on the sample data of the first omics includes: classifying each patient in the sample data of the first omics according to prognosis or a disease stage of each patient, wherein a classification result includes at least two categories.

Exemplarily, assuming that a predicted result of an algorithm model according to the embodiment of the present disclosure is prognosis of a patient, then, when data preprocessing is performed, for each patient in sample data, a quantity of total survival years is calculated from a quantity of survival days, and the patient is classified according to the quantity of total survival years. For example, when the quantity of total survival years of the patient is less than or equal to 2 years, the patient is determined to be in category I; when the quantity of total survival years of the patient is greater than 2 years, the patient is determined to be in category II. After categories of all patients in the sample data have been determined, the determined patient categories are added to a last column of the methylation data set. In this example, patients are classified into two categories according to the quantity of total survival years, however, in other examples, patients may be classified into three or more categories as desired, which is not limited in the embodiment of the present disclosure.

Exemplarily, assuming that a predicted result of an algorithm model according to the embodiment of the present disclosure is a disease stage of a patient, then, when data preprocessing is performed, for each patient in sample data, the patient is classified according to the disease stage of the patient, for example, clinical stage categories may be directly used as categories I, II, and III, etc. After categories of all patients in the sample data have been determined, the determined patient categories are added to a last column of the methylation data set. According to the embodiment of the present disclosure, patients may be classified into two, three, or more categories according to disease stages of the patients, which is not limited in the embodiment of the present disclosure.

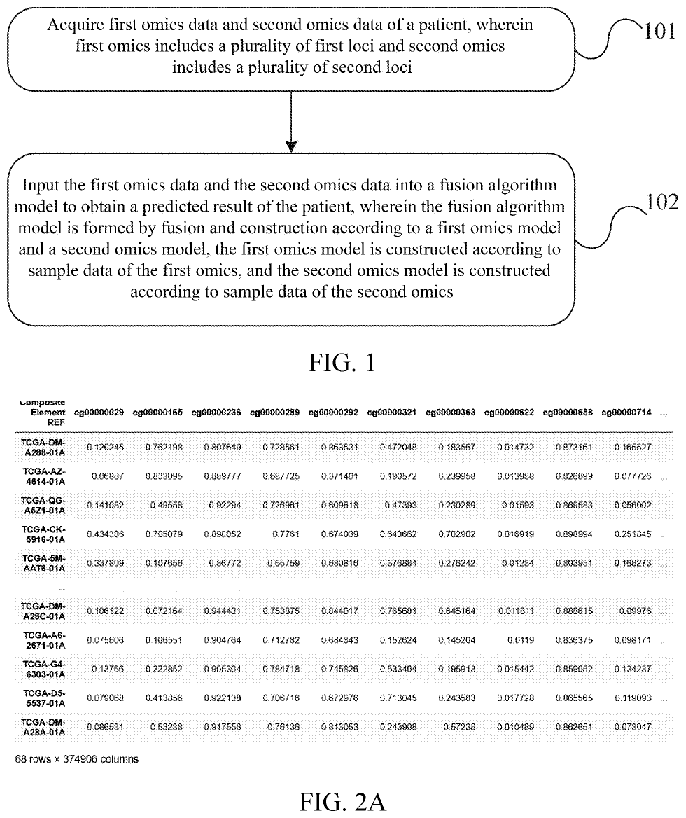

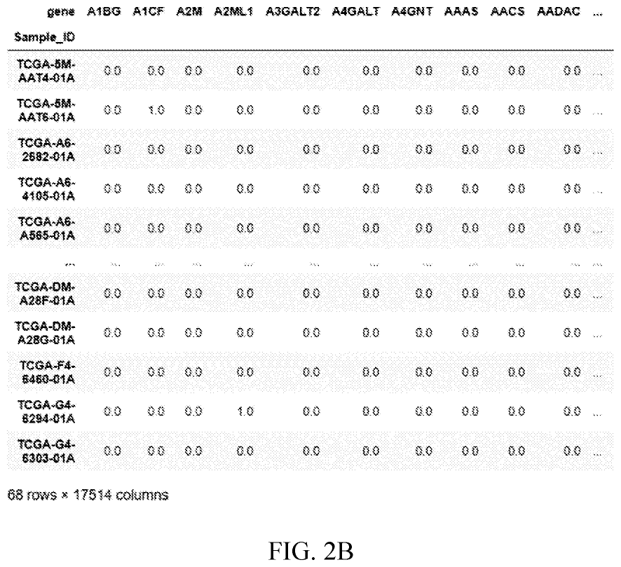

Exemplarily, it is assumed that after data preprocessing, the methylation data set includes 68 patient samples and 374,905 methylation loci, as shown in FIG. 2A, among them, row names represent different patients, columns 1 to 374,905 represent different methylation loci, and column 374,906 represent patient categories.

In some exemplary implementation modes, the second omics model is constructed according to the sample data of the second omics, which may include: performing data preprocessing on the sample data of the second omics; performing feature filtering on the sample data of the second omics; calculating a second similarity matrix between the sample data of the second omics, and constructing a second undirected link network according to the calculated second similarity matrix; and constructing the second omics model according to the constructed second undirected link network and eigenvalues of the second omics.

In some exemplary implementation modes, a second locus may be a gene locus, however, which is not limited in the embodiment of the present disclosure.

In some exemplary implementation modes, performing data preprocessing on the sample data of the second omics may include: screening a set type of gene mutation, for each patient sample, marking it as 1 if the patient sample has a gene mutation on a certain gene, and marking it as 0 if there is no gene mutation, and forming a gene mutation data set; or, counting a quantity of mutations of the patient sample on a certain gene and marking it as n, and forming a gene mutation data set.

Exemplarily, it is assumed that after data preprocessing, the gene mutation data set includes 68 patient samples and 17,513 gene loci, as shown in FIG. 2B, among them, row names represent different patients, columns 1 to 17,513 represent different gene loci, and column 17,514 represents patient categories. Patient categories in the gene mutation data set are the same as corresponding patient categories in the methylation data set.

In some exemplary implementation modes, performing feature filtering on the sample data of the first omics may include: calculating a variance of data of each first locus; comparing the calculated variance with a preset first variance threshold; and deleting a first locus whose calculated variance is less than the preset first variance threshold.

In some exemplary implementation modes, performing feature filtering on the sample data for the second omics may include: calculating a variance of data of each second locus; comparing the calculated variance with a preset second variance threshold; and deleting a second locus whose calculated variance is less than the preset second variance threshold.

In the disease analysis method according to the embodiment of the present disclosure, candidate features that may optimize performance of a subsequent model are determined through feature filtering. Magnitudes of a preset first variance threshold and a preset second variance threshold may be set according to actual situations of a data set of first loci and a data set of second loci. Exemplarily, a magnitude of the preset first variance threshold may be set such that after a first locus whose calculated variance is smaller than the preset first variance threshold is deleted, a quantity of remaining first loci accounts for 0.5% to 2% of a quantity of first loci before deletion. Similarly, a magnitude of the preset second variance threshold may be set such that after a second locus whose calculated variance is smaller than the preset second variance threshold is deleted, a quantity of remaining second loci accounts for 0.5% to 2% of a quantity of second loci before deletion. A preset threshold of the quantity of first loci and a preset threshold of the quantity of second loci may be dynamically adjusted according to accuracy of a final model.

Exemplarily, still taking a case where a first locus is a DNA methylation locus and a second locus is a gene locus as an example, a variance is calculated for each column of methylation locus data in a methylation data set, and a methylation locus with low variance characteristic is filtered according to a preset first variance threshold. A variance is calculated for each column of gene mutation situations of a gene locus mutation data set, and a gene locus with low variance characteristic is filtered according to a preset second variance threshold.

A methylation locus or gene locus with a variance less than a preset variance threshold (including the preset first variance threshold or the preset second variance threshold) is filtered, which may be represented using following formula 1.

$${Column}_{a} = \{ \begin{matrix}δ {n}_{i} \geq ε , retained \\ δ {n}_{i} < ε , discarded\end{matrix} \qquad ( formula 1 )$$

δnᵢ is a variance of methylation values or a variance of gene mutation values of all patients at a specific methylation locus or gene locus, ε is the preset first variance threshold or the preset second variance threshold, and when δnᵢ is greater than or equal to ε, the methylation locus or gene locus is retained; when δnᵢ is less than ε, the methylation locus or gene locus is discarded, and Columnₐ is a remaining feature column after filtering.

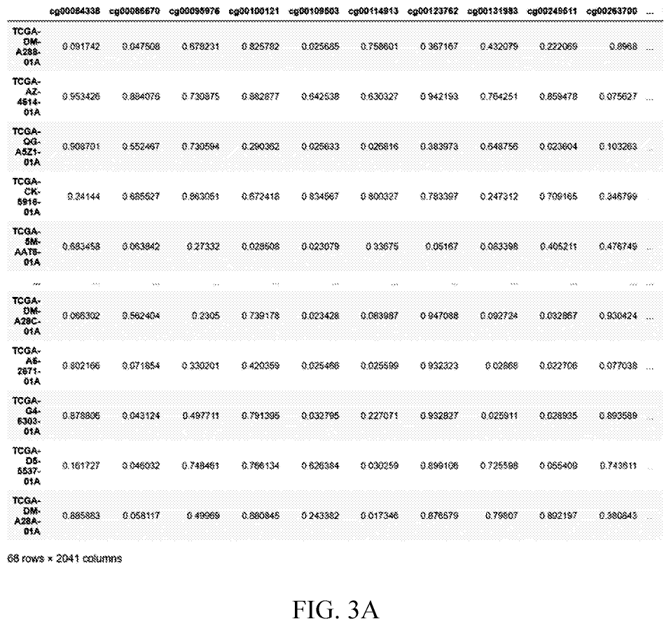

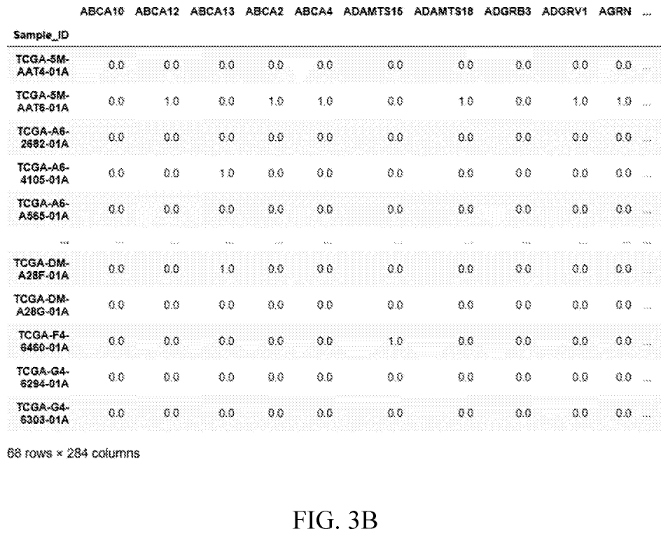

Exemplarily, still taking the aforementioned methylation data set and gene mutation data set as an example, the methylation data set includes 68 patient samples and 2040 methylation loci after low variance feature filtering, as shown in FIG. 3A. The gene mutation data set includes 68 patient samples and 283 gene loci, as shown in FIG. 3B.

In some exemplary implementation modes, performing feature filtering on the sample data of the first omics may further include: selecting a base model, training for many times using the base model and the sample data of the first omics, and removing x first loci in the sample data of the first omics at the end of each training, wherein weight values of the x first loci are lower x weight values among weight values of all first loci obtained by each training, and x is a natural number greater than or equal to 1, until a quantity of remaining first loci is equal to a preset threshold of a quantity of first loci.

In some exemplary implementation modes, the base model may be a linear regression model, a logistic regression model, or a decision tree model.

In some exemplary implementation modes, performing feature filtering on the sample data of the second omics may further include: selecting a base model, training for many times using the base model and the sample data of the second omics, and removing y second loci in the sample data of the second omics at the end of each training, wherein weight values of the y second loci are lower y weight values among weight values of all second loci obtained by each training, and y is a natural number greater than or equal to 1, until a quantity of remaining second loci is equal to a preset threshold of a quantity of second loci.

Exemplarily, still taking a case where a first locus is a DNA methylation locus and a second locus is a gene locus as an example, for the methylation loci after feature filtering above, a class of model algorithm is selected as the base model, a fitting data set is traversed, and a methylation locus with lower contribution to the model (i.e., a lower weight) is recursively filtered out until a quantity of remaining methylation loci is equal to a preset threshold of a quantity of first loci. For the gene loci after feature filtering above, a class of model algorithm is selected as the base model, a fitting data set is traversed, and a gene locus with lower contribution to the model (i.e., a lower weight) is recursively filtered out until a quantity of remaining gene loci is equal to a preset threshold of a quantity of second loci. The preset threshold of the quantity of first loci and the preset threshold of the quantity of second loci may be set according to a quantity of first loci after low variance feature filtering and a quantity of second loci after low variance feature filtering. Exemplarily, a magnitude of the preset threshold of the quantity of first loci may be set to be more than 40% of the quantity of first loci after low variance feature filtering; a magnitude of the preset threshold of the quantity of second loci may be set to be more than 40% of the quantity of second loci after low variance feature filtering. The preset threshold of the quantity of first loci and the preset threshold of the quantity of second loci may be dynamically adjusted according to accuracy of a final model.

A methylation locus or gene locus with lower contribution to the model (i.e., a lower weight) is filtered, which may be represented using following formula 2.

$${Column}_{b} = Sort ( S\_ \{ i \} ) , i = 1 , 2 , 3 … \qquad ( formula 2 )$$

S_{i} is each feature subset. After sorting feature variables in importance (i.e. weights), N features with higher importance are screened, and then a feature subset is constructed until a length of the feature subset is equal to a set quantity of features. Column_{b} is a remaining feature column after further filtering on a basis of Columnₐ.

According to the embodiment of the present disclosure, accuracy of a final result can be effectively improved (running time after feature filtering is reduced and accuracy is improved) through a feature filtering algorithm dominated by low variance filtering and recursive dimension reduction.

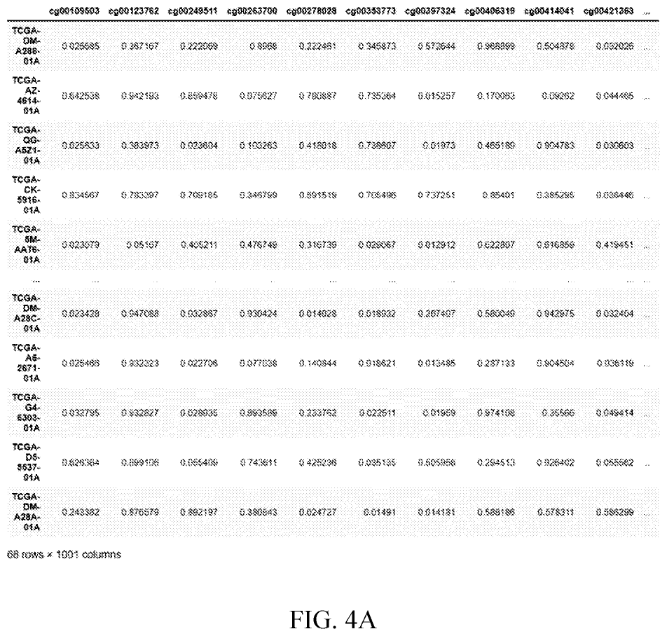

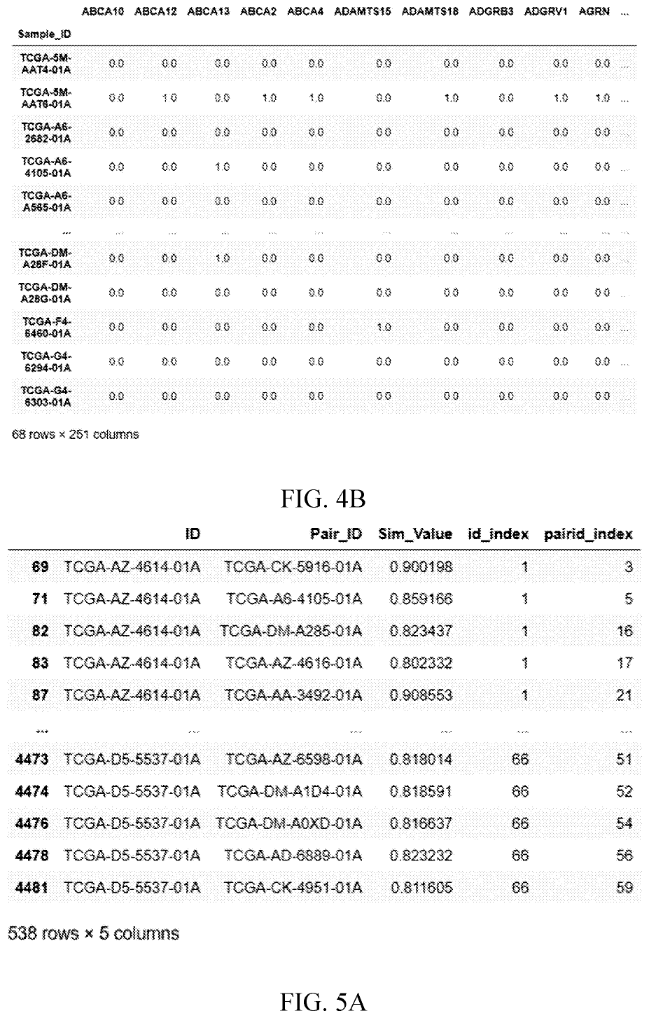

Exemplarily, still taking the aforementioned methylation data set and gene mutation data set as an example, the methylation data set includes 68 patient samples and 1,000 methylation loci after recursive dimension reduction feature filtering, as shown in FIG. 4A. The gene mutation data set includes 68 patient samples and 250 gene loci, as shown in FIG. 4B.

In some exemplary implementation modes, the first similarity matrix between the sample data of the first omics or the second similarity matrix between the sample data of the second omics may be calculated using a cosine algorithm. a similarity score between patient A and patient B is calculated using the cosine algorithm, which may be expressed using following formula 3.

$$cos ( θ ) = \frac{{\sum }_{i = 1}^{n} ( {A}_{i} \times {B}_{i} )}{\sqrt{{\sum }_{i = 1}^{n} {( {A}_{i} )}^{2}} \times \sqrt{{\sum }_{i = 1}^{n} {( {B}_{i} )}^{2}}} \qquad ( formula 3 )$$

Herein, Aᵢ is a methylation level value at a certain methylation locus or a mutation situation at a certain gene locus of patient A, Bᵢ is a methylation level value at a same locus or a mutation situation at a same gene of patient B, and n is a quantity of all methylation loci or a quantity of all genes.

According to the similarity score (i.e., a cosine value cos(B)), a difference between patient A and patient B is measured. The closer cos(B) is to 1, the more similar patient A and patient B are; the closer cos(B) is to 0, the less similar patient A and patient B are.

Exemplarily, assuming that a similarity threshold of the methylation data set is set to 0.8 according to a statistic situation of similarity scores of the methylation loci, when a similarity score of methylation loci between two patients is greater than or equal to 0.8, it is considered that there is a network relationship between the two patients; when the similarity score of the methylation loci between two patients is less than 0.8, it is considered that there is no network relationship between the two patients, thus a first undirected link network is established.

Exemplarily, assuming that a similarity threshold of the gene mutation data set is set to 0.5 according to a statistic situation of similarity scores of gene loci, when a similarity score of gene loci between two patients is greater than or equal to 0.5, it is considered that there is a network relationship between the two patients; when the similarity score of the gene loci between two patients is less than 0.5, it is considered that there is no network relationship between the two patients, thus a second undirected link network is established.

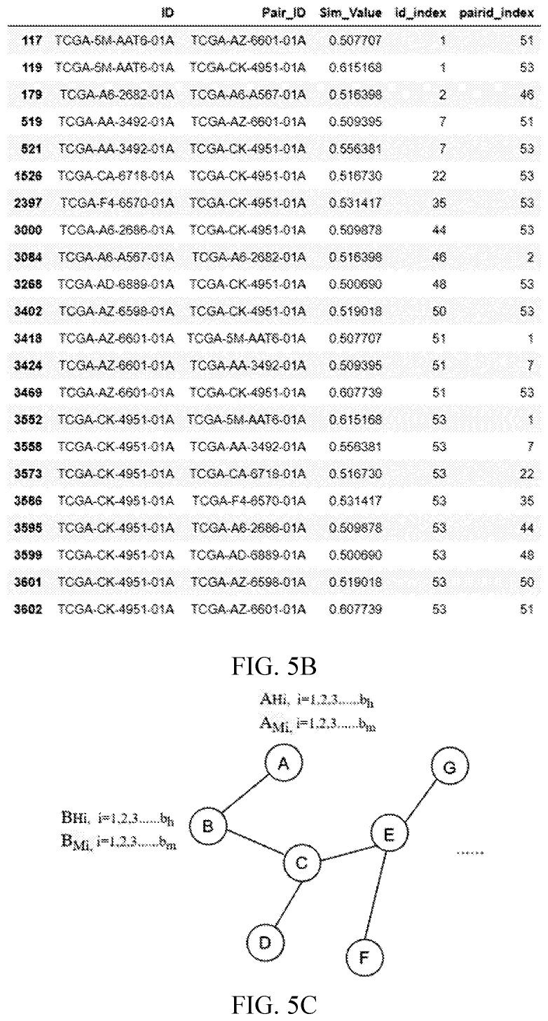

Based on a data set constructed in FIG. 3A, an established first undirected link network relationship data set is shown in FIG. 5A, and based on a data set constructed in FIG. 3B, an established second undirected link network relationship data set is shown in FIG. 5B.

The first similarity matrix or the second similarity matrix may be established according to following formula 4, wherein lᵢ and lⱼ are data in row i (patient i) and data in row j (patient j) in the methylation data set or gene locus data set, respectively, and i≠j, and S(lᵢ, lⱼ) represents a similarity score between patient i and patient j in the methylation data set or gene locus data set.

$${X}_{i j} = \{ \begin{matrix}S ( {l}_{i} , {l}_{j} ) , if S ( {l}_{i} , {l}_{j} ) \geq ε \\ 0\end{matrix} \qquad ( formula 4 )$$

When the similarity score S(lᵢ, lⱼ) is greater than a set similarity threshold ε, there is a network relationship between patient i and patient j, otherwise there is no network relationship. A network diagram of the first undirected link network or the second undirected link network after construction is shown in FIG. 5C. Herein, A, B, C . . . are patient numbers, A_{Hi} is a methylation value of patient A at methylation locus i, methylation loci include 1, 2, 3, . . . , bₕ, A_{Mi} is a gene mutation situation of patient A at gene locus i, and gene loci include 1, 2, 3, . . . , bₘ.

In some exemplary implementation modes, the first undirected link network or the second undirected link network may be constructed according to formula 5 as follows.

$${f}_{i}^{( l + 1 )} = σ ( {\sum }_{j ϵ {N}_{i}} \frac{1}{{c}_{ij}} {f}_{j}^{( l )} ) \qquad ( formula 5 )$$

Herein, fᵢ^{(l)} is a characteristic of node i at layer l, σ is a nonlinear activation function, Cᵢⱼ is a normalization factor, and N, is a neighboring node of node i.

In this embodiment, the first undirected link network is constructed according to the first similarity matrix, and the second undirected link network is constructed according to the second similarity matrix, and both the first undirected link network and the second undirected link network are two-dimensional undirected link networks. According to the embodiment of the present disclosure, an algorithm model is comprehensively constructed by considering characteristic of a node itself and characteristic of a neighboring node through the two-dimensional undirected link networks, and performance of the model is evaluated using a multi-fold cross-verification method.

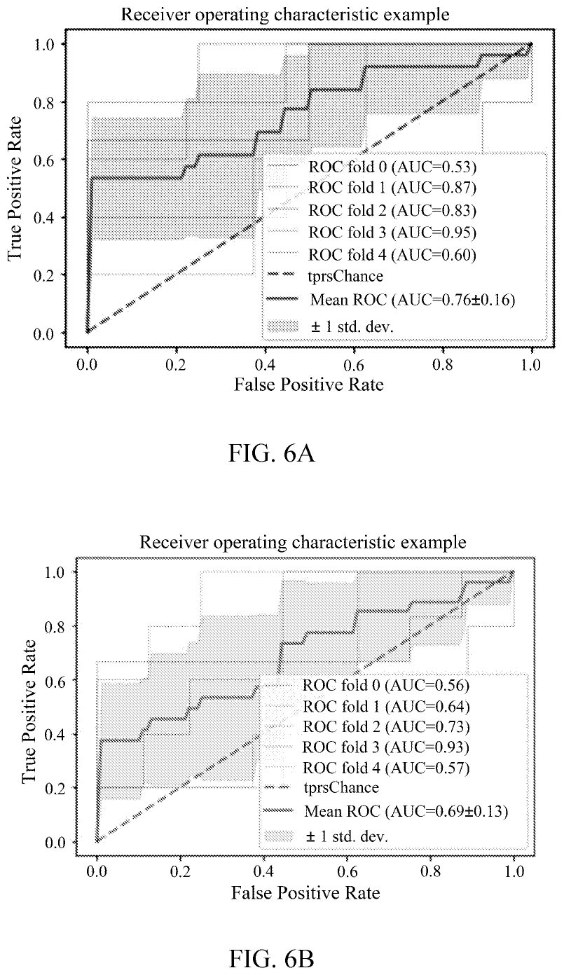

Exemplarily, a methylation algorithm model is constructed according to the first undirected link network relationship data set of FIG. 5A, and a ROC curve graph obtained is shown in FIG. 6A. It may be found that an Area Under roc Curve (AUC) median value of the methylation algorithm model is 0.76 after 5-fold cross-validation. A gene mutation algorithm model is constructed according to the second undirected link network relationship data set of FIG. 5B, and a ROC curve graph obtained is shown in FIG. 6B. It may be found that an AUC median value of the gene mutation algorithm model is 0.69 after 5-fold cross-validation.

A Receiver Operating Characteristic (ROC) curve is also referred as a sensitivity curve. A reason for this name is that various points on the curve reflect same sensitivity, and they are all responses to same signal stimulus, but only results obtained under several different determination criteria. The receiver operating characteristic curve is a coordinate graph composed of False Positive Rate as a horizontal axis and True Positive Rate as a vertical axis, and a curve drawn by different results obtained by subjects under specific stimulus conditions due to different determination criteria. A value of an Area Under roc Curve (AUC) is a size of an area below the ROC curve. A larger AUC represents better performance. Usually, when AUC=0.5, it shows that this diagnostic method has no diagnostic value. When AUC is within (0.5, 0.7], it shows that the diagnostic method has relatively low accuracy, when AUC is within (0.7, 0.9], it shows that the diagnostic method has certain accuracy, and when AUC is greater than 0.9, it shows that the diagnostic method has relatively high accuracy.

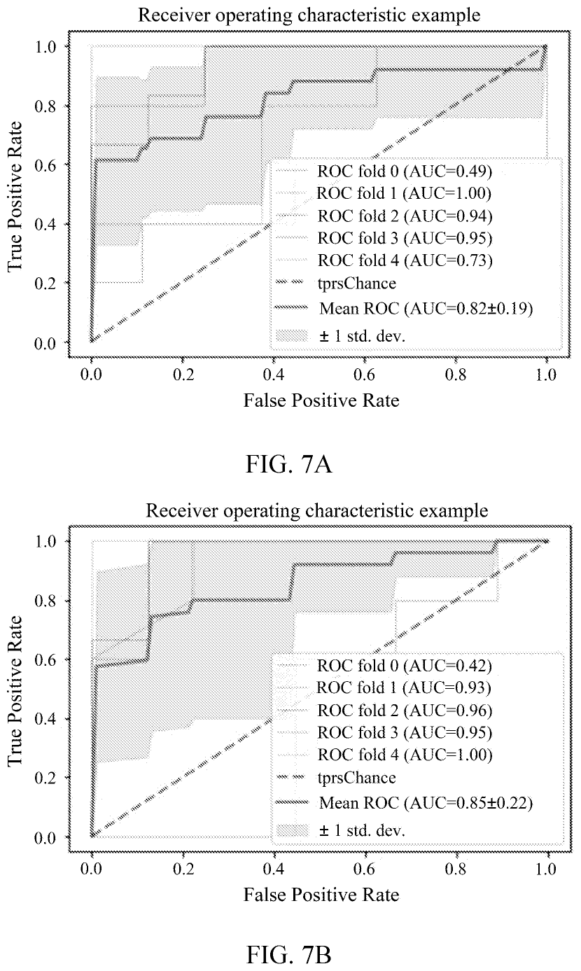

Exemplarily, still taking the aforementioned methylation data set and gene mutation data set as an example, according to a methylation data set after secondary feature screening (low variance filtering and recursive dimension reduction) of FIG. 4A, a first undirected link network relationship data set is established, and then a methylation algorithm model is constructed. A ROC curve graph obtained is shown in FIG. 7A. It may be found that after 5-fold cross-validation, an AUC median value of the methylation algorithm model is 0.82, and it may be seen that performance of the model after secondary filtering is obviously better than that after first filtering. According to a gene mutation data set after secondary feature screening (low variance filtering and recursive dimension reduction) of FIG. 4B, a second undirected link network relationship data set is established, and then a gene mutation algorithm model is constructed. A ROC curve graph obtained is shown in FIG. 7B. It may be found that an AUC median value of the gene mutation algorithm model is 0.85 after 5-fold cross-validation, and similarly, performance of the model after secondary filtering is obviously better than that of first filtering. At the same time, due to reduction of a quantity of features of methylation loci and gene loci, a time cost of constructing an algorithm model after feature filtering is further reduced.

In some exemplary implementation modes, the fusion algorithm model is formed by fusion and construction according to the first omics model and the second omics model, which may include: multiplying probability values with a same predicted category in the first omics model and the second omics model to obtain a product probability matrix; and constructing the fusion algorithm model according to a two-dimensional undirected link network and the product probability matrix, wherein the two-dimensional undirected link network may be the first undirected link network or the second undirected link network.

In this embodiment, on a basis of completing construction of the methylation algorithm model and the gene mutation algorithm model, the two types of models may be further fused to form the fusion algorithm model. The fusion algorithm model integrates multiple models into a final model, which is superior to any single model in model performance.

Exemplarily, still taking a case where the first locus is a DNA methylation locus and the second locus is a gene locus as an example, probability values with a same predicted category of the methylation algorithm model and the gene mutation algorithm model are multiplied to form a product probability matrix, wherein a width of the matrix is a quantity of required predicted categories.

Assuming that a quantity of predicted categories includes two categories, the product probability matrix is calculated according to following formula 6.

$${X}_{i} = [ \prod p ( {P}_{i}^{T , I} ) , \prod p ( {P}_{i}^{T , II} ) \qquad ( formula 6 )$$

Herein, Πp(pᵢ^{T,I}) is a probability product of a probability of predicting a prognosis category of a patient as class I in the methylation algorithm model and a probability of predicting a prognosis category of a patient as class I in the gene mutation algorithm model, and Πp(Pᵢ^{T,II}) is a probability product of a probability of predicting a prognosis category of a patient as class II in the methylation algorithm model and a probability of predicting a prognosis category of a patient as class II in the gene mutation algorithm model. It needs to be pointed out that the width of the matrix increases according to increase of a quantity of categories, such as [Πp(pᵢ^{T,I}), Πp(pᵢ^{T,II}), Πp(pᵢ^{T,III}) . . . .

An obtained product probability matrix is used as new eigenvalues of each patient, replacing a methylation level value of a methylation locus and an original feature of a gene mutation situation in a two-dimensional link network. A fusion algorithm model is constructed based on the first undirected link network or the second undirected link network previously constructed, and performance of the model is evaluated using a cross-validation method.

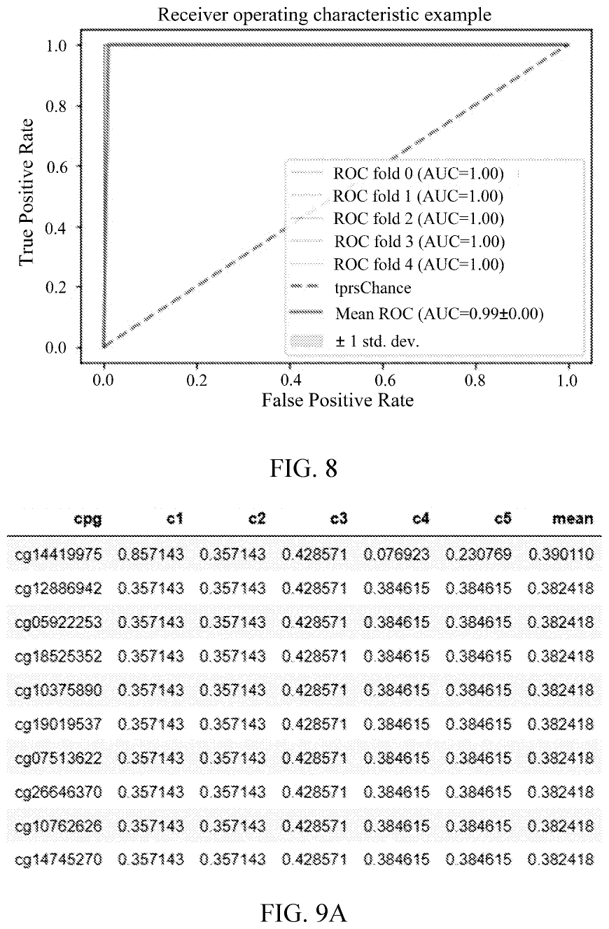

Exemplarily, still taking the aforementioned methylation data set and gene mutation data set as an example, a ROC curve graph of a fusion algorithm model is shown in FIG. 8. It may be found that an AUC median value of the fusion algorithm model is 0.99 after 5-fold cross-validation, indicating that performance of the fusion algorithm model is better than that of the methylation algorithm model and the gene mutation algorithm model alone after secondary filtering.

In some exemplary implementation modes, the disease analysis method may further include: performing following operations for a plurality of first loci one by one: randomly shuffling sample data of a first locus currently selected, combining the randomly shuffled sample data of the first locus with sample data of another first locus to form new eigenvalues of first omics, and reconstructing a first omics model according to the new eigenvalues of the first omics and a first undirected link network; evaluating a mean absolute error between a predicted result and a true result of the reconstructed first omics model; and using a randomly shuffled first locus corresponding to first N1 larger mean absolute errors as a candidate molecular marker, wherein N1 is a natural number greater than or equal to 1.

In the embodiment of the present disclosure, the candidate molecular marker refers to a molecular marker most related to occurrence and development of a disease in clinic, and may be a biological substance such as a gene, a methylation locus, and protein, etc., which has better reference value in diagnosis and treatment of the disease.

In some exemplary implementation modes, the disease analysis method may further include: performing following operations for a plurality of the first loci one by one: randomly shuffling sample data of a first locus currently selected, combining the randomly shuffled sample data of the first locus with sample data of another first locus to form new eigenvalues of first omics, and reconstructing a first omics model for K times through K-fold cross-validation; evaluating a mean absolute error between a predicted result and a true result of the reconstructed K first omics models, and calculating an average value of K mean absolute errors; and using a randomly shuffled first locus corresponding to an average value of first N1 larger K mean absolute errors as a candidate molecular marker, wherein N1 is a natural number greater than or equal to 1 and K is a natural number greater than 1.

In some exemplary implementation modes, an average value of K mean absolute errors may be calculated according to formula 7 as follows.

$${A}_{l} = \frac{1}{nK} {\sum }_{i = 1}^{n} \vert {y\_pred}_{shu ffle ( l )} - y\_true \vert \qquad ( formula 7 )$$

Herein, y_true is a true value, y_pred_{shuffle_(l)} is a predicted result after randomly shuffling column l in a data set, n is a total quantity of predicted results, and K is a quantity of cross-validation partition folds of the data set.

In some exemplary implementation modes, the disease analysis method may further include: performing following operations for a plurality of second loci one by one: randomly shuffling sample data of a second locus currently selected, combining the randomly shuffled sample data of the second locus with sample data of another second locus to form new eigenvalues of second omics, and reconstructing a second omics model according to the new eigenvalues of the second omics and a second undirected link network; evaluating a mean absolute error between a predicted result and a true result of the reconstructed second omics model; and using a randomly shuffled second locus corresponding to first N2 larger mean absolute errors as a candidate molecular marker, wherein N2 is a natural number greater than or equal to 1.

In some exemplary implementation modes, the disease analysis method may further include: performing following operations for a plurality of second loci one by one: randomly shuffling sample data of a second locus currently selected, combining the randomly shuffled sample data of the second locus with sample data of another second locus to form new eigenvalues of second omics, and reconstructing a second omics model for K times through K-fold cross-validation; evaluating a mean absolute error between a predicted result and a true result of the reconstructed K second omics models, and calculating an average value of K mean absolute errors; and using a randomly shuffled second locus corresponding to an average value of first N2 larger K mean absolute errors as a candidate molecular marker, wherein N2 is a natural number greater than or equal to 1 and K is a natural number greater than 1.

In some exemplary implementation modes, the average value of K mean absolute errors may be calculated according to formula 7 as follows.

$${A}_{l} = \frac{1}{nK} {\sum }_{i = 1}^{n} \vert {y\_pred}_{shu ffle ( l )} - y\_true \vert \qquad ( formula 7 )$$

Herein, y_true is a true value, y_pred_{shuffle (l)} is a predicted result after randomly shuffling column l in a data set, n is a total quantity of predicted results, and K is a quantity of cross-validation partition folds of the data set.

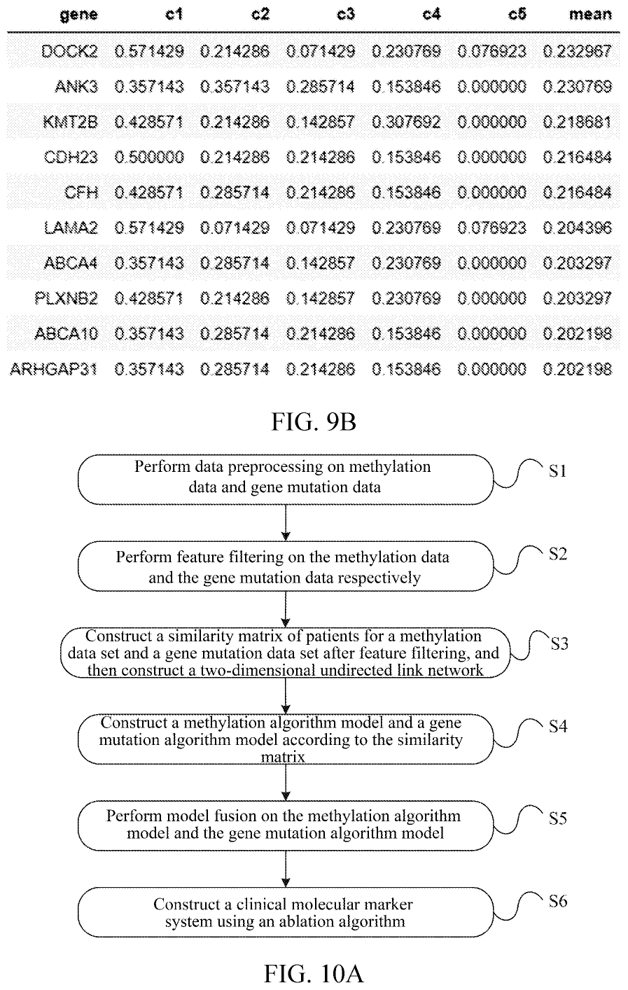

Exemplarily, still taking the aforementioned methylation data set and gene mutation data set as an example, according to the aforementioned disease analysis method, a clinical molecular marker system of top 10 of methylation is obtained and shown in FIG. 9A, and a clinical molecular marker system of top 10 of gene mutation is as shown in FIG. 9B, herein, c1, c2, c3, c4, and c5 are mean absolute error values obtained by five times of cross-validation respectively, mean is an average value of five mean absolute error values, and methylation loci involved are cg14419975, cg12886942, cg05922253, cg18525352, cg10375890, cg19019537, cg07513622, cg26646370, cg10762626, and cg14745270. Genes involved are DOCK2, ANK3, KMT2B, CDH23, CFH, LAMA2, ABCA4, PLXNB2, ABCA10, and ARHGAP31.

Technical solutions of the embodiment of the present disclosure is described in detail below by taking a case where a first locus is a DNA methylation locus and a second locus is a gene locus in the disease analysis method according to the embodiment of the present disclosure as an example.

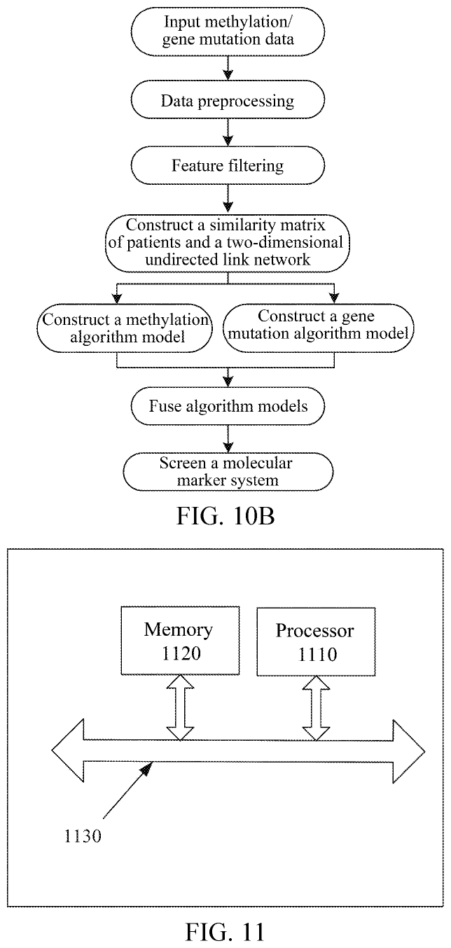

In the disease analysis method according to the embodiment of the present disclosure, a clinical molecular marker system and a systematic algorithm for diagnosis and treatment of a tumor patient may be constructed by comprehensively analyzing a DNA methylation locus and a gene locus, as shown in FIGS. 10A and 10B. The method mainly includes following acts.

S1: performing data preprocessing on data such as DNA methylation and gene mutation of a patient and corresponding total survival time of the patient.

S2: performing feature filtering on eigenvalues such as methylation loci/genes of DNA methylation/gene mutation data respectively.

S3: constructing a similarity matrix of the patient through the eigenvalues of DNA methylation/gene mutation data, and constructing a two-dimensional link network with the patient as a node according to a similarity score, using a similarity algorithm respectively.

S4: constructing an algorithm model including input feature nodes, middle layer nodes, and output result nodes by taking the two-dimensional link network of the patient and corresponding eigenvalues as input objects, and evaluating accuracy of the algorithm model.

S5: performing feature integration for each classification prediction probability of the algorithm model of DNA methylation and gene mutation omics data, and fusing two omics to construct an algorithm model, and evaluating accuracy of the algorithm model, and comparing accuracy of a fusion algorithm model and accuracy of a monoomics algorithm model of DNA methylation/gene mutation.

S6: evaluating a loss value of each feature of DNA methylation/gene mutation after random shuffle using an ablation algorithm, and selecting several types of features with higher loss values as a clinical molecular marker system.

In the disease analysis method according to the embodiment of the present disclosure, prognosis of a patient can be determined more accurately and a local network of patients with internal connection can be established. Each act is described in detail below.

S1: performing data preprocessing on methylation data and gene mutation data.

S1.1: deleting a methylation locus when there is one or more patient samples at the locus where no methylation level value is detected, or retaining more than 80% of methylation loci where there is data for patients, if there is missing data, filling with a median or average value to obtain final methylation loci and form a methylation data set.

S1.2: Screening a set type of gene mutation, for each patient sample, marking it as 1 if the patient sample has a mutation on a certain gene, and marking it as 0 if there is no mutation, and forming a gene mutation data set; or, counting a quantity of mutations of the patient sample on a certain gene and marking it as n, and forming a gene mutation data set.

S1.3: converting prognosis of a patient into a digital type, and constructing two kinds of data sets: methylation-prognosis and gene mutation-prognosis.

S2: performing feature filtering on methylation data and gene mutation data respectively.

S2.1: calculating a variance for each column of methylation locus data in the methylation data set, and filtering methylation loci with low variance characteristics according to a set specific threshold.

S2.2: calculating a variance for each column of gene mutation situations in the gene mutation data set, and filtering genes with low variance characteristics according to a set specific threshold.

Optionally, the filtering method is as follows:

$${Column}_{a} = \{ \begin{matrix}δ {n}_{i} \geq ε , retained \\ δ {n}_{i} < ε , discard\end{matrix} \qquad ( formula 1 )$$

Herein, δnᵢ is a variance of methylation values or gene mutation values of all patients at a specific methylation locus or gene, F is a set variance threshold, when δnᵢ is greater than or equal to the set variance threshold, the methylation locus or gene is retained, and Columnₐ is a remaining feature column after filtering.

S2.3: optionally, for methylation loci after filtering in S2.1, a class of model algorithm is selected as a base model, and a fitting data set is traversed to recursively filter out a methylation locus with lower contribution to the model (i.e., a lower weight) until a quantity of remaining methylation loci is equal to a preset quantity of features.

S2.4: optionally, for genes after filtering in S2.2, a class of model algorithm is selected as a base model, and a fitting data set is traversed to recursively filter out a gene locus with lower contribution to the model (i.e., a lower weight) until a quantity of remaining gene loci is equal to a preset quantity of features.

Optionally, a filtering method is as follows.

$${Column}_{b} = Sort ( S\_ \{ i \} ) , i = 1 , 2 , 3 , … \qquad ( formula 2 )$$

Herein, S_{i} is each feature subset. After sorting feature variables in importance, N features with higher importance are screened, and then a feature subset is constructed until a length of the feature subset is equal to a set quantity of features. Column_{b} is a remaining feature column after further filtering on a basis of Columnₐ.

S3: constructing a similarity matrix of patients for a methylation data set and a gene mutation data set after feature filtering, and then constructing a two-dimensional undirected link network with patients as nodes.

Similarity between any two rows of patient data in the methylation or gene mutation data set is calculated. When a similarity value is greater than a set threshold, it is considered that there is a network relationship between two patients. Based on this method, a two-dimensional undirected link network is constructed.

Optionally, a calculation method is as follows.

A similarity score between every two patients in the methylation or gene mutation data set is calculated using a cosine algorithm, as shown in formula 3.

$$cos ( θ ) = \frac{{\sum }_{i = 1}^{n} ( {A}_{i} \times {B}_{i} )}{\sqrt{{\sum }_{i = 1}^{n} {( {A}_{i} )}^{2}} \times \sqrt{{\sum }_{i = 1}^{n} {( {B}_{i} )}^{2}}} \qquad ( formula 3 )$$

Herein, A, is a methylation level value at a certain methylation locus or a mutation situation at a certain gene locus of patient A, Bᵢ is a methylation level value at a same locus or a mutation situation at a same gene of patient B, and n is a quantity of all methylation loci or a quantity of all gene loci.

$${X}_{ij} = \{ \begin{matrix}S ( {l}_{i} , {l}_{j} ) , if S ( {l}_{i} , {l}_{j} ) \geq ε \\ 0\end{matrix} \qquad ( formula 4 )$$

Herein, Iᵢ and Iⱼ are any two rows of patient data, and i≠j. When a similarity score S(Iᵢ,Iⱼ) is greater than a set threshold ε, there is a network relationship between patient i and patient j, otherwise there is no network relationship. A network diagram after construction is shown in FIG. 5C. Herein, A, B, C . . . are patient numbers, A_{Hi} is a methylation value of patient A at methylation locus i, methylation loci include 1, 2, 3, . . . , bₕ, A_{Mi} is a gene mutation situation of patient A at gene locus i, and gene loci include 1, 2, 3, . . . , bₘ.

S4: constructing a methylation algorithm model and a gene mutation algorithm model according to the similarity matrix.

In this act, characteristics of two-dimensional undirected link network nodes itself and characteristics of neighboring nodes are considered, an algorithm model is comprehensively constructed, and performance of the model is evaluated using a cross-validation method.

Optionally, a calculation method is as follows.

$${f}_{i}^{( l + 1 )} = σ ( {\sum }_{j ϵ {N}_{i}} \frac{1}{{C}_{ij}} {f}_{j}^{( l )} ) \qquad ( formula 5 )$$

Herein, fᵢ^{(l)} is a characteristic of node i at layer l, σ is nonlinear activation, Cᵢⱼ is a normalization factor, and Nᵢ is a neighboring node of node i.

S5: performing model fusion on the methylation algorithm model and the gene mutation algorithm model.

S5.1: multiplying probability values of a same predicted category of the methylation algorithm model and the gene mutation algorithm model to form a product probability matrix, wherein a width of the matrix is a quantity of required predicted categories.

Optionally, a calculation method is as follows.

$${X}_{i} = [ \prod p ( {P}_{i}^{T , I} ) , \prod p ( {P}_{i}^{T , II} ) \qquad ( formula 6 )$$

Herein, Πp(pᵢ^{T,I}) is a probability product of a predicted result being class I in the methylation algorithm model and the gene mutation algorithm model, and Πp(pᵢ^{T,II}) is a probability product of a predicted result being class II in the methylation algorithm model and the gene mutation algorithm model. It needs to be pointed out that the width of the matrix increases according to increase of a quantity of categories, such as [Πp(pᵢ^{T,I}),Πp(pᵢ^{T,II}),Πp(pᵢ^{T,III}) . . . .

S5.2: taking an obtained product probability matrix as new eigenvalues of each patient, and constructing a fusion algorithm model according to a model construction method in S4 based on the two-dimensional undirected link network of patients previously constructed in S3 (which may be the two-dimensional undirected link network of patients constructed according to the methylation data set or the two-dimensional undirected link network of patients constructed according to the gene mutation data set), and evaluating performance of the model using a cross-validation method.

S6: constructing a clinical molecular marker system using an ablation algorithm.

In order, methylation level values of methylation loci or gene mutation situation data of genes in each column are randomly shuffled, a mean absolute error between a predicted result after shuffling and a true result is evaluated, and first N methylation loci or gene loci with larger mean absolute errors after shuffling are screened as candidate molecular markers.

Optionally, a calculation method is as follows.

$${A}_{l} = \frac{1}{nK} {\sum }_{i = 1}^{n} \vert {y\_pred}_{shu ffle ( l )} - y\_true \vert \qquad ( formula 7 )$$

Herein, y_true is a true value, y_pred_{shuffle (l)} is a predicted result after randomly shuffling column l in a data set, n is a total quantity of predicted results, and K is a quantity of cross-validation partition folds of the data set.

The technical solutions of the embodiment of the present disclosure will be further described in detail below by taking an actual methylation data set and gene mutation data set as an example.

In terms of methylation, a methylation locus is deleted when there is one or more patient samples at the locus where no methylation level value is detected, and remaining loci are retained.

A methylation data set after screening is shown in FIG. 2A, with 68 patient samples and 374,905 methylation loci. For prognosis data of a patient, total survival years are obtained by calculating survival days. When a quantity of years is less than or equal to 2 years, prognosis of the patient is determined as class I, and when the quantity of years is greater than 2 years, the prognosis of the patient is determined as class II. A processed prognosis category is added to a last column of the data set.

A low variance threshold is set to 0.08, a variance of each column of the methylation data set is calculated, and a methylation locus with a variance less than 0.08 is deleted. A methylation data set after deletion is shown in FIG. 3A. After screening, there are 68 patient samples and 2,040 methylation loci.

Based on the data set constructed above, a similarity score between every two patients is calculated using a cosine algorithm. Optionally, a calculation method is as follows.

$$cos ( θ ) = \frac{{\sum }_{i = 1}^{n} ( {A}_{i} \times {B}_{i} )}{\sqrt{{\sum }_{i = 1}^{n} {( {A}_{i} )}^{2}} \times \sqrt{{\sum }_{i = 1}^{n} {( {B}_{i} )}^{2}}}$$

Herein, Aᵢ is a methylation level value at a certain methylation locus of patient A, Bᵢ is a methylation level value of patient B at a same locus, and n is equal to a quantity of all methylation loci.

According to a statistic situation of similarity scores, a threshold of the methylation data set is set to 0.8. When a score of two patients is greater than or equal to 0.8, it is considered that there is a network relationship between the two patients. An obtained network relationship data set is shown in FIG. 5A. According to the aforementioned method, a two-dimensional link undirected network graph is constructed. Based on a two-dimensional link undirected network, with methylation level value situations as features, a methylation algorithm model is constructed according to the aforementioned method, and performance of the methylation algorithm model is evaluated using a cross-validation method.

A ROC curve graph of the methylation algorithm model is shown in FIG. 6A. It may be found that an AUC median value of the methylation algorithm model is 0.76 after 5-fold cross-validation.

In terms of gene mutation, for each patient sample, if the sample has a mutation on a certain gene, it is marked as 1, and if there is no mutation, it is marked as 0.

A gene mutation data set after screening is shown in FIG. 2B, with 68 patient samples and 17,513 gene loci. For prognosis data of a patient, total survival years are obtained by calculating survival days. When a quantity of years is less than or equal to 2 years, prognosis of the patient is determined as class I, and when the quantity of years is greater than 2 years, the prognosis of the patient is determined as class II. A processed prognosis category is added to a last column of the data set.

A low variance threshold is set to 0.08, a variance of each column of the gene mutation data set is calculated, and a gene with a variance less than 0.08 is deleted. A gene mutation data set after deletion is shown in FIG. 3B. After screening, there are 68 patient samples and 283 genes. It may be seen that for each patient sample, due to secondary classification marking according to whether a gene is mutated or not, majority of data of some genes are 0, a variance is relatively small, and vast majority of loci are filtered.

Based on the data set constructed above, a similarity score between every two patients is calculated using a cosine algorithm. Optionally, a calculation method is as follows.

$$cos ( θ ) = \frac{{Σ}_{i = 1}^{n} ( {A}_{i} \times {B}_{i} )}{\sqrt{{{Σ}_{i = 1}^{n} ( {A}_{i} )}^{2}} \times \sqrt{{{Σ}_{i - - 1}^{n} ( {B}_{i} )}^{2}}}$$

Herein, Aᵢ is a mutation situation of patient A in a certain gene, Bᵢ is a mutation situation of patient B in a same gene, and n is equal to a quantity of all genes.

According to a statistic situation of similarity scores, a threshold of the gene mutation data set is set to 0.5. When a score of two patients is greater than or equal to 0.5, it is considered that there is a network relationship between the two patients. An obtained network relationship data set is shown in FIG. 5B. According to the aforementioned method, a two-dimensional link undirected network graph is constructed. Based on a two-dimensional link undirected network, with gene mutation situations as features, a gene mutation algorithm model is constructed according to the aforementioned method, and performance of the gene mutation algorithm model is evaluated using a cross-validation method.

A ROC curve graph of the gene mutation algorithm model is shown in FIG. 6B. It may be found that an AUC median value of the gene mutation algorithm model is 0.69 after 5-fold cross-validation.

When features of the methylation data set and the gene mutation data set are filtered, further feature screening may be carried out on a basis of the aforementioned embodiment, a linear regression method is selected as a base model, a quantity of features required by the methylation data set is set to be 1000, and a quantity of features required by the gene mutation data set is set to be 250. After recursively deleting features, remaining features are used for fitting the base model, and statistics on accuracy of the model is made to determine which feature combinations have highest contribution to performance of the model. According to a following formula, a following operation is performed.

$${Column}_{b} = Sort ( S\_ \{ i \} ) , i = 1 , 2 , 3 , … , a$$

On a basis of performing feature screening of a data set for one time in the aforementioned embodiment, secondary feature screening is performed on feature subsets of a methylation locus and a gene locus respectively. A methylation data set after the secondary feature screening is shown in FIG. 4A. After screening, there are 68 patient samples and 1,000 methylation loci. A gene mutation data set after the secondary feature screening is shown in FIG. 4B. After screening, there are 68 patient samples and 250 genes.

Based on the data set constructed above, methylation loci or gene locus situations between every two patients are calculated using a cosine algorithm, and a similarity between every two patients is obtained. According to a statistic situation of similarity scores, a two-dimensional link undirected network graph is constructed. Based on a two-dimensional link undirected network, with methylation level values or gene mutation value situations as features, a methylation algorithm model or a gene mutation algorithm model is constructed according to the aforementioned method (formula 5), and performance of the methylation algorithm model or gene mutation algorithm model is evaluated using a cross-validation method.

Among them, a ROC curve graph of the methylation algorithm model is shown in FIG. 7A. It may be found that an AUC median value of the methylation algorithm model is 0.82 after 5-fold cross-validation, and it may be seen that performance of the model after secondary filtering is obviously better than that of first filtering. A ROC curve graph of the gene mutation algorithm model is shown in FIG. 7B. It may be found that an AUC median value of the gene mutation algorithm model is 0.85 after 5-fold cross-validation, and similarly, performance of the model after secondary filtering is obviously better than that of first filtering. At the same time, due to reduction of a quantity of methylation loci and gene features, a time cost of constructing an algorithm model after feature filtering is further reduced.

On a basis of completing construction of the methylation algorithm model and the gene mutation algorithm model, two types of models may be further fused. Optionally, according to the following formula, following operations are performed.

$${X}_{i} = [ \prod p ( {P}_{i}^{T , I} ) , \prod p ( {P}_{i}^{T , II} )$$

A probability of predicting prognosis of a patient as class I in the methylation algorithm model is multiplied with a probability of predicting a prognosis category of a patient as class I in the gene mutation algorithm model, and a probability of predicting prognosis of a patient as class II in the methylation algorithm model is multiplied with a probability of predicting a prognosis category of a patient as class II in the gene mutation algorithm model. Then, numerical matrices of class I and II are used as features to replace original features of methylation level values of methylation loci and gene mutation situations in a two-dimensional link network, and a fusion algorithm model is further constructed, and the model is evaluated using 5-fold cross-validation.

A ROC curve graph of the fusion algorithm model is shown in FIG. 8. It may be found that an AUC median value of the fusion algorithm model is 0.99 after 5-fold cross-validation, indicating that performance of the fusion algorithm model is better than that of the methylation algorithm model and the gene mutation algorithm model alone after secondary filtering.

Further, on a basis of the aforementioned embodiment, a molecular marker system is determined, 1,000 methylation locus data in the methylation data set or 250 gene mutation situation data in the gene mutation data set are traversed and shuffled to replace a feature matrix in a two-dimensional link undirected network, and similarly, an algorithm model is constructed using a 5-fold cross-validation method, mean absolute errors between all predicted values and true values of the model after each column of methylation locus data or gene mutation situation data are shuffled are evaluated. Then, mean absolute errors of five types of data with different splitting modes are averaged again, and top 10 methylation loci or genes with larger final values are taken as a clinical molecular marker system. Optionally, a calculation method is as follows.

$${A}_{l} = \frac{1}{nK} {\sum }_{i = 1}^{n} \vert {y\_pred}_{shu ffle ( l )} - y\_true \vert$$

Herein, a clinical molecular marker system of top 10 of methylation is shown in FIG. 9A, a clinical molecular marker system of top 10 of gene mutation is shown in FIG. 9B, c1, c2, c3, c4, and c5 are mean absolute error values obtained by five times of cross-validation respectively, and mean is an average value of five mean absolute error values. Methylation loci involved are cg14419975, cg12886942, cg05922253, cg18525352, cg10375890, cg19019537, cg07513622, cg26646370, cg10762626, and cg14745270. Genes involved are DOCK2, ANK3, KMT2B, CDH23, CFH, LAMA2, ABCA4, PLXNB2, ABCA10, and ARHGAP31.

In the disease analysis method according to the embodiment of the present disclosure, prognosis of a patient can be more accurately determined through cross-integration analysis of DNA methylation and gene mutation; a systematic patient network is established through a calculation method of a two-dimensional link network, similar diagnosis and treatment schemes for patients in a local network can be implemented in a targeted manner and prognosis of patients can be determined more effectively; accuracy of a final result may be effectively improved (running time after feature filtering is reduced and accuracy is improved) through a feature filtering algorithm dominated by low variance filtering and recursive dimension reduction.

An embodiment of the present disclosure further provides a disease analysis apparatus, including a memory and a processor connected to the memory, the memory is configured to store instructions, the processor is configured to perform acts of a disease analysis method according to any embodiment of the present disclosure based on the instructions stored in the memory.

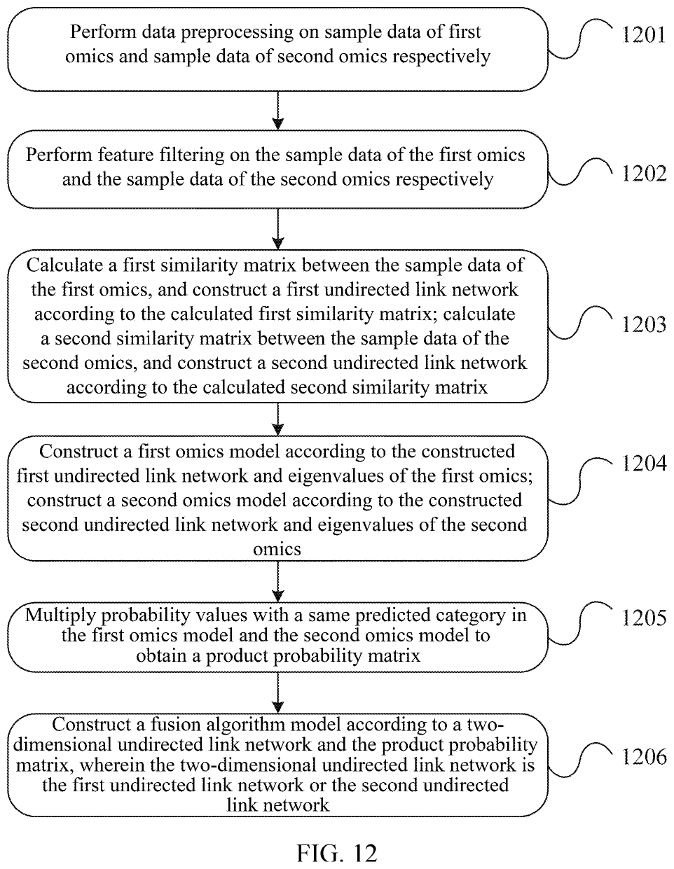

As shown in FIG. 11, in one example, the disease analysis apparatus may include a processor 1110, a memory 1120, and a bus system 1130, wherein the processor 1110 and the memory 1120 are connected via the bus system 1130, the memory 1120 is configured to store instructions, the processor 1110 is configured to execute the instructions stored by the memory 1120 to acquire first omics data and second omics data of a patient, wherein first omics includes a plurality of first loci and second omics includes a plurality of second loci; input the first omics data and the second omics data into a fusion algorithm model to obtain a predicted result of the patient, wherein the fusion algorithm model is formed by fusion and construction according to a first omics model and a second omics model, the first omics model is constructed according to sample data of the first omics, and the second omics model is constructed according to sample data of the second omics.

It should be understood that the processor 1110 may be a Central Processing Unit (CPU), and the processor 1110 may also be another general-purpose processor, a Digital Signal Processor (DSP), an Application Specific Integrated Circuit (ASIC), a Field Programmable Gate Array (FPGA) or another programmable logic device, a discrete gate or a transistor logic device, and a discrete hardware component, etc. The general-purpose processor may be a microprocessor, or the processor may be any conventional processor, etc.

The memory 1120 may include a read only memory and a random access memory, and provides instructions and data to the processor 1110. A portion of the memory 1120 may also include a non-volatile random access memory. For example, the memory 1120 may also store information of a device type.

The bus system 1130 may include a power bus, a control bus, a status signal bus, or the like in addition to a data bus. However, for clarity of illustration, various buses are all denoted as the bus system 1130 in FIG. 11.

In an implementation process, processing performed by a processing device may be completed through an integrated logic circuit of hardware in the processor 1110 or instructions in a form of software. That is, acts of the methods in the embodiments of the present disclosure may be embodied as executed and completed through a hardware processor, or executed and completed through a combination of hardware in the processor and a software module. The software module may be located in a storage medium such as a random access memory, a flash memory, a read only memory, a programmable read only memory, or an electrically erasable programmable memory, or a register, etc. The storage medium is located in the memory 1120. The processor 1110 reads information in the memory 1120, and completes the acts of the above method in combination with its hardware. In order to avoid repetition, detailed description is not provided herein.

An embodiment of the present disclosure also provides a computer-readable storage medium having stored thereon a computer program, when the program is executed by a processor, the disease analysis method according to any embodiment of the present disclosure is implemented. A method of driving prognosis analysis by executing executable instructions is substantially the same as the disease analysis method provided in the above embodiments of the present disclosure and will not be repeated here.

In some possible implementation modes, various aspects of the disease analysis method provided in the present disclosure may also be implemented in a form of a program product, which includes a program code. When the program product is run on a computer device, the program code is used for enabling the computer device to execute acts in the disease analysis method according to various exemplary implementation modes of the present disclosure described above in this specification, for example, the computer device may execute the disease analysis method described in the embodiments of the present disclosure.

For the program product, any combination of one or more readable media may be adopted. A readable medium may be a readable signal medium or a readable storage medium. The readable storage medium may be, for example, but is not limited to, an electrical, magnetic, optical, electromagnetic, infrared, or semiconductor system, apparatus, or device, or a combination of any of the above. A more specific example (a non-exhaustive list) of the readable storage medium includes: an electrical connection with one or more wires, a portable disk, a hard disk, a Random Access Memory (RAM), a Read Only Memory (ROM), an Erasable Programmable Read Only Memory (EPROM or a flash memory), an optical fiber, a portable Compact Disk Read Only Memory (CD-ROM), an optical storage device, a magnetic storage device, or any suitable combination of the above.

As shown in FIG. 12, an embodiment of the present disclosure also provides a training method of a disease analysis model, which includes following acts.

Act 1201: performing data preprocessing on sample data of first omics and sample data of second omics respectively.

Act 1202: performing feature filtering on the sample data of the first omics and the sample data of the second omics respectively.

Act 1203: calculating a first similarity matrix between the sample data of the first omics, and constructing a first undirected link network according to the calculated first similarity matrix; calculating a second similarity matrix between the sample data of the second omics, and constructing a second undirected link network according to the calculated second similarity matrix.

Act 1204: constructing a first omics model according to the constructed first undirected link network and eigenvalues of the first omics; constructing a second omics model according to the constructed second undirected link network and eigenvalues of the second omics.

Act 1205: multiplying probability values with a same predicted category in the first omics model and the second omics model to obtain a product probability matrix.

Act 1206: constructing a fusion algorithm model according to a two-dimensional undirected link network and the product probability matrix, wherein the two-dimensional undirected link network is the first undirected link network or the second undirected link network.

In some exemplary implementation modes, the training method may further include: performing following operations for a plurality of first loci one by one: randomly shuffling sample data of a first locus currently selected, combining the randomly shuffled sample data of the first locus with sample data of another first locus to form new eigenvalues of the first omics, and reconstructing a first omics model; evaluating a mean absolute error between a predicted result of the reconstructed first omics model and a true result; and using a randomly shuffled first locus corresponding to first N1 larger mean absolute errors as a candidate molecular marker, wherein N1 is a natural number greater than or equal to 1.

In some exemplary implementation modes, the training method may further include: performing following operations for a plurality of first loci one by one: randomly shuffling sample data of a first locus currently selected, combining the randomly shuffled sample data of the first locus with sample data of another first locus to form new eigenvalues of first omics, and reconstructing a first omics model for K times through K-fold cross-validation; evaluating a mean absolute error between a predicted result of reconstructed K first omics models and a true result, and calculating an average value of K mean absolute errors; and using a randomly shuffled first locus corresponding to an average value of first N1 larger K mean absolute errors as a candidate molecular marker, wherein N1 is a natural number greater than or equal to 1 and K is a natural number greater than 1.

In some exemplary implementation modes, the training method may further include: performing following operations for a plurality of second loci one by one: randomly shuffling sample data of a second locus currently selected, combining the randomly shuffled sample data of the second locus with sample data of another second locus to form new eigenvalues of second omics, and reconstructing a second omics model according to the new eigenvalues of the second omics and a second undirected link network; evaluating a mean absolute error between a predicted result of the reconstructed second omics model and a true result; and using a randomly shuffled second locus corresponding to first N2 larger mean absolute errors as a candidate molecular marker, wherein N2 is a natural number greater than or equal to 1.

In some exemplary implementation modes, the training method may further include: performing following operations for a plurality of second loci one by one: randomly shuffling sample data of a second locus currently selected, combining the randomly shuffled sample data of the second locus with sample data of another second locus to form new eigenvalues of second omics, and reconstructing a second omics model for K times through K-fold cross-validation; evaluating a mean absolute error between a predicted result of reconstructed K second omics models and a true result, and calculating an average value of K mean absolute errors; and using a randomly shuffled second locus corresponding to an average value of first N2 larger K mean absolute errors as a candidate molecular marker, wherein N2 is a natural number greater than or equal to 1 and K is a natural number greater than 1.

An embodiment of the present disclosure also provides a training apparatus of a disease analysis model, including a memory and a processor connected to the memory, wherein the memory is configured to store instructions, the processor is configured to execute acts of the training method of the disease analysis model according to any embodiment of the present disclosure based on the instructions stored in the memory.

An embodiment of the present disclosure also provides a computer-readable storage medium having stored thereon a computer program, when the program is executed by a processor, the training method of the disease analysis model according to any embodiment of the present disclosure is implemented.

In some possible implementation modes, various aspects of the training method of the disease analysis model according to the present disclosure may also be implemented in a form of a program product, which includes a program code. When the program product is run on a computer device, the program code is used for enabling the computer device to execute acts in the training method of the disease analysis model according to various exemplary implementation modes of the present disclosure described above in this specification, for example, the computer device may execute the training method of the disease analysis model described in the embodiments of the present disclosure.

For the program product, any combination of one or more readable media may be adopted. A readable medium may be a readable signal medium or a readable storage medium. The readable storage medium may be, for example, but is not limited to, an electrical, magnetic, optical, electromagnetic, infrared, or semiconductor system, apparatus, or device, or a combination of any of the above. A more specific example (a non-exhaustive list) of the readable storage medium includes: an electrical connection with one or more wires, a portable disk, a hard disk, a Random Access Memory (RAM), a Read Only Memory (ROM), an Erasable Programmable Read Only Memory (EPROM or a flash Memory), an optical fiber, a portable Compact Disk Read Only Memory (CD-ROM), an optical storage device, a magnetic storage device, or any suitable combination of the above.

It may be understood by those of ordinary skills in the art that all or some acts in the method and function modules/units in the system and the apparatus disclosed above may be implemented as software, firmware, hardware, and appropriate combinations thereof. In a hardware implementation mode, division of the function modules/units mentioned in the above description is not always corresponding to division of physical components. For example, one physical component may have multiple functions, or a function or an act may be executed by several physical components in cooperation. Some components or all components may be implemented as software executed by a processor such as a digital signal processor or a microprocessor, or implemented as hardware, or implemented as an integrated circuit such as an application specific integrated circuit. Such software may be distributed in a computer-readable medium, and the computer-readable medium may include a computer storage medium (or a non-transitory medium) and a communication medium (or a transitory medium). As known to those of ordinary skills in the art, the term computer storage medium includes volatile and nonvolatile, and removable and irremovable media implemented in any method or technology for storing information (for example, computer-readable instructions, a data structure, a program module, or other data). The computer storage medium includes, but is not limited to, a RAM, a ROM, an EEPROM, a flash memory or another memory technology, CD-ROM, a Digital Versatile Disk (DVD) or another optical disk storage, a magnetic cassette, a magnetic tape, a magnetic disk storage or another magnetic storage apparatus, or any other medium that may be configured to store desired information and may be accessed by a computer. In addition, it is known to those of ordinary skills in the art that the communication medium usually includes computer-readable instructions, a data structure, a program module, or other data in a modulated data signal of, such as, a carrier or another transmission mechanism, and may include any information delivery medium.

Although the implementation modes disclosed in the present disclosure are described as above, the described contents are only implementation modes which are used for facilitating understanding of the present disclosure, but are not intended to limit the present disclosure. Any skilled person in the art to which the present disclosure pertains may make any modification and variation in forms and details of implementation without departing from the spirit and scope of the present disclosure. However, the patent protection scope of the present disclosure should be subject to the scope defined in the appended claims.

1. A disease analysis method, comprising:
acquiring first omics data and second omics data of a patient, wherein first omics comprises a plurality of first loci and second omics comprises a plurality of second loci; and
inputting the first omics data and the second omics data into a fusion algorithm model to obtain a predicted result of the patient, wherein the fusion algorithm model is formed by fusion and construction according to a first omics model and a second omics model, the first omics model is constructed according to sample data of the first omics, and the second omics model is constructed according to sample data of the second omics.

2. The disease analysis method according to claim 1, wherein a first locus is a DeoxyriboNucleic Acid (DNA) methylation locus and a second locus is a gene locus; or, the first locus is a gene locus and the second locus is a DNA methylation locus; wherein the gene locus comprises information of a gene mutation situation and/or information of a gene expression situation.

3. The disease analysis method according to claim 1, wherein the first omics model is constructed according to the sample data of the first omics, comprising:
performing data preprocessing on the sample data of the first omics;
performing feature filtering on the sample data of the first omics;
calculating a first similarity matrix between the sample data of the first omics, and constructing a first undirected link network according to the calculated first similarity matrix; and
constructing the first omics model according to the constructed first undirected link network and eigenvalues of the first omics.

4. The disease analysis method according to claim 3, wherein the performing data preprocessing on the sample data of the first omics comprises:
deleting one or more first loci in the sample data of the first omics, wherein there is no data value for at least one patient sample at each deleted first locus.

5. The disease analysis method according to claim 3, wherein the performing data preprocessing on the sample data of the first omics comprises:
deleting one or more first loci in the sample data of the first omics, wherein there is no data value for at least b % of patient samples at each deleted first locus, and b is a real number greater than 0; and
performing data filling on a patient sample with no data value in the sample data of the first omics.

6. The disease analysis method according to claim 3, wherein the performing data preprocessing on the sample data of the first omics comprises: classifying each patient in sample data of the first omics and the second omics according to prognosis or a disease stage of each patient, wherein a classification result comprises at least two categories.

7. The disease analysis method according to claim 3, wherein the performing feature filtering on the sample data of the first omics comprises:
calculating a variance for data of each of the first loci;
comparing the calculated variance with a preset first variance threshold; and
deleting a first locus with a calculated variance less than the preset first variance threshold.

8. The disease analysis method according to claim 7, wherein the performing feature filtering on the sample data of the first omics further comprises:
selecting a base model, training for multiple times using the base model and the sample data of the first omics, removing x first loci in the sample data of the first omics at the end of each training, wherein weight values of the x first loci are lower x weight values among weight values of all the first loci obtained in each training, and x is a natural number greater than or equal to 1, until a quantity of remaining first loci is equal to a preset threshold of a quantity of first loci.

9. The disease analysis method according to claim 8, wherein the base model is any one of following: a linear regression model, a logistic regression model, or a decision tree model.

10. The disease analysis method according to claim 1, wherein the second omics model is constructed according to the sample data of the second omics, comprising:
performing data preprocessing on the sample data of the second omics;
performing feature filtering on the sample data of the second omics;
calculating a second similarity matrix between the sample data of the second omics, and constructing a second undirected link network according to the calculated second similarity matrix; and
constructing the second omics model according to the constructed second undirected link network and eigenvalues of the second omics.

11. The disease analysis method according to claim 10, wherein the fusion algorithm model is formed by fusion and construction according to the first omics model and the second omics model, comprising:
multiplying probability values with a same predicted category in the first omics model and the second omics model to obtain a product probability matrix; and
constructing the fusion algorithm model according to a two-dimensional undirected link network and the product probability matrix, wherein the two-dimensional undirected link network is a first undirected link network established according to the sample data of the first omics or a second undirected link network established according to the sample data of the second omics.

12. The disease analysis method according to claim 1, further comprising:
performing following operations for the plurality of first loci one by one: randomly shuffling sample data of a first locus currently selected, combining the randomly shuffled sample data of the first locus with sample data of another first locus to form new eigenvalues of the first omics, and reconstructing a first omics model; evaluating a mean absolute error between a predicted result and a true result of the reconstructed first omics model; and
using a randomly shuffled first locus corresponding to first N1 larger mean absolute errors as a candidate molecular marker, wherein N1 is a natural number greater than or equal to 1.

13. The disease analysis method according to claim 1, further comprising:
performing following operations for the plurality of first loci one by one: randomly shuffling sample data of a first locus currently selected, combining the randomly shuffled sample data of the first locus with sample data of another first locus to form new eigenvalues of the first omics, and reconstructing a first omics model for K times through K-fold cross-validation; evaluating a mean absolute error between a predicted result and a true result of reconstructed K first omics models, and calculating an average value of K mean absolute errors; and
using a randomly shuffled first locus corresponding to an average value of first N1 larger K mean absolute errors as a candidate molecular marker, wherein N1 is a natural number greater than or equal to 1 and K is a natural number greater than 1.

14. The disease analysis method according to claim 1, further comprising:
performing following operations for the plurality of second loci one by one: randomly shuffling sample data of a second locus currently selected, combining the randomly shuffled sample data of the second locus with sample data of another second locus to form new eigenvalues of the second omics, and reconstructing a second omics model; evaluating a mean absolute error between a predicted result and a true result of the reconstructed second omics model; and
using a randomly shuffled second locus corresponding to first N2 larger mean absolute errors as a candidate molecular marker, wherein N2 is a natural number greater than or equal to 1.

15. The disease analysis method according to claim 1, further comprising:
performing following operations for the plurality of second loci one by one: randomly shuffling sample data of a second locus currently selected, combining the randomly shuffled sample data of the second locus with sample data of another second locus to form new eigenvalues of the second omics, and reconstructing a second omics model for K times through K-fold cross-validation; evaluating a mean absolute error between a predicted result and a true result of reconstructed K second omics models, and calculating an average value of K mean absolute errors; and
using a randomly shuffled second locus corresponding to an average value of first N2 larger K mean absolute errors as a candidate molecular marker, wherein N2 is a natural number greater than or equal to 1 and K is a natural number greater than 1.

16. A disease analysis apparatus, comprising a memory and a processor connected to the memory, wherein the memory is configured to store instructions, and the processor is configured to execute acts of the disease analysis method according to claim 1 based on the instructions stored in the memory.

17. A non-transitory computer-readable storage medium having stored thereon a computer program, wherein when the program is executed by a processor, the disease analysis method according to claim 1 is implemented.

18. A training method of a disease analysis model, comprising:
performing data preprocessing on sample data of first omics and sample data of second omics respectively;
performing feature filtering on the sample data of the first omics and the sample data of the second omics respectively;
calculating a first similarity matrix between the sample data of the first omics, and constructing a first undirected link network according to the calculated first similarity matrix;
calculating a second similarity matrix between the sample data of the second omics, and constructing a second undirected link network according to the calculated second similarity matrix;
constructing a first omics model according to the constructed first undirected link network and eigenvalues of the first omics; constructing a second omics model according to the constructed second undirected link network and eigenvalues of the second omics;
multiplying probability values with a same predicted category in the first omics model and the second omics model to obtain a product probability matrix; and
constructing a fusion algorithm model according to a two-dimensional undirected link network and the product probability matrix, wherein the two-dimensional undirected link network is the first undirected link network or the second undirected link network.

19. A training apparatus of a disease analysis model, comprising a memory and a processor connected to the memory, wherein the memory is configured to store instructions, and the processor is configured to execute acts of the training method of the disease analysis model according to claim 18 based on the instructions stored in the memory.

20. A non-transitory computer-readable storage medium having stored thereon a computer program, wherein when the program is executed by a processor, the training method of the disease analysis model according to claim 18 is implemented.
